(19) United States
(12) Patent Application Publication
(10) Pub. No.: US 2024/0074699 A1
(43) Pub. Date: Mar. 7, 2024

(54) System and Method for Quantification and Feedback of Eye Deviations

(71) Applicant: New Jersey Institute of Technology, Newark, NJ (US)

(72) Inventors: Christopher Morris, Franklin Lakes, NJ (US); Tara Lynn Alvarez, Whippany, NJ (US); Mitchell Scheiman, Bala Cynwyd, PA (US); Chang Yaramothu, Hackensack, NJ (US); John Vito d'Antonio-Bertagnolli, Mount Laurel, NJ (US)

(73) Assignee: New Jersey Institute of Technology, Newark, NJ (US)

(21) Appl. No.: 18/243,354

(22) Filed: Sep. 7, 2023

Related U.S. Application Data
(60) Provisional application No. 63/404,325, filed on Sep. 7, 2022.

(51) Int. Cl. A61B 5/00; A61B 3/00; A61B 3/08; A61B 3/113

(52) U.S. Cl. CPC A61B 5/4863; A61B 3/0025; A61B 3/0041; A61B 3/085; A61B 3/113; A61B 5/4076; A61B 5/4836; A61B 5/486; A61B 5/6803; A61B 5/6814; A61B 5/7271; A61B 5/7405; A61B 5/742; A61B 5/7455; A61B 5/746; A61B 2505/07; A61B 2560/0223; A61B 2560/0406; A61B 2560/0443; A61B 2560/0462

(57) ABSTRACT

Disclosed is a system, method, and apparatus for quantification and feedback of eye deviations during normal viewing conditions. A sensory biofeedback system serves to emit a sensory cue such as a tone or vibration when a certain threshold of eye deviation has been exceeded. The biofeedback system allows objective monitoring of the accuracy of eye alignment that can be used outside of a clinical setting or inside a clinical setting.

### CROSS-REFERENCE TO RELATED APPLICATIONS

The present application claims the benefit of the filing date of U.S. Provisional Application No. 63/404,325 filed Sep. 7, 2022, the disclosure of which is hereby incorporated herein by reference.

### STATEMENT REGARDING FEDERALLY SPONSORED RESEARCH

This invention was made with government support under contract grant number EY023261 awarded by the National Institutes of Health. The government has certain rights in the invention.

### FIELD OF THE DISCLOSURE

The present disclosure relates to assessing an ocular misalignment disorder while a patient performs daily living activities. In particular, the present disclosure relates to a system and method for quantification and biofeedback of eye alignment deviations during normal daily living viewing conditions.

### BACKGROUND

Binocular vision (eye alignment) disorders are common in children and adults affecting 15% to 20% of the population. Strabismus is the term reserved for a binocular vision disorder which is severe enough to cause a manifest misalignment of the eyes (one eye turned in, out, up, or down). Strabismus can be constant (one eye always misaligned) or intermittent (eye misaligned only a percentage of the time). In addition to a loss of depth perception and potentially a loss of acuity in the misaligned eye, strabismus causes a cosmetic issue which may lead to social problems if not treated successfully.

The two main categories of strabismus are esotropia (one eye crosses in) and exotropia (one eye drifts out). Another way of classifying strabismus is to describe whether it is a constant misalignment or intermittent misalignment. For example, esotropia or exotropia can be constant or intermittent.

There are three significant challenges for eyecare professionals when managing strabismus. Two are diagnostic and the third is a treatment challenge and the apparatus we have developed helps the clinician with all of these clinical dilemmas. Intermittent strabismus has been shown to be a variable condition with unpredictable inter-day, intraday, and even minute-by-minute fluctuations. This variability complicates everyday decision-making by clinicians trying to determine if the strabismus has deteriorated to the level at which intervention should be considered. Traditional clinical measures are brief assessments completed during a time-limited examination and may not reflect the true nature of eye misalignment throughout the day.

Recognizing these challenges, prior state of the art investigators developed “subjective” visual measures called the “office-control score”. These measures can assess the size or magnitude of a deviation but they cannot capture any changes in magnitude over time as they can only be assessed over a short period of time by a trained clinician. In addition, smaller strabismic deviations (less than 10-15Δ) cannot be detected by simply visual inspection from the clinician. The second challenge is determining whether treatment has been successful. Because of the variability of strabismus, it is difficult to assess the effectiveness of surgical or nonsurgical treatment because the examination takes place for only short periods of time. The eyes may look aligned during the short examination, but other times of the day the eyes may be misaligned.

The third challenge, among many addressed by the present invention, is related to the effectiveness of the treatment of strabismus. Treatment of strabismus includes both surgical and non-surgical (functional) methods. Strabismus surgery has a reasonably good success rate in achieving a cosmetic improvement (eyes look straight even though a small deviation may be present) in cases of constant esotropia, but it is not as successful in achieving a functional cure (normal alignment of and depth perception). The success rates with intermittent exotropia are not as good and there is also a relatively high rate of recurrence of intermittent exotropia after surgery. As a result, non-surgical management of intermittent exotropia is often required.

Nonsurgical methods used for treatment of strabismus include occlusion (covering one eye), lenses, prism, and vision rehabilitation. There is evidence that vision therapy is an effective treatment for improving control of intermittent exotropia. Biofeedback has been shown to enable a patient to quickly learn to converge and diverge the eyes and this can shorten the amount of time needed for therapeutic intervention.

Biofeedback is a technique based on conditioning principles and has been used in a variety of medical applications. The technique provides a person with immediate information regarding a biological process, which is normally beyond their conscious awareness, thus facilitating voluntary regulation of this same function. In the case of strabismus, a person is generally unaware of when the eyes are aligned or misaligned. In biofeedback therapy for strabismus, the present invention monitors eye alignment in real time and sends a sensory stimulus to the person (auditory or tactile) that makes the person immediately aware of eye position. This awareness can lead to better control while the feedback is provided and afterwards when the feedback is eliminated.

Using this feedback, patients with strabismus can regain control of the ocular misalignment in shorter periods of time than when engaged in traditional vision therapy. Nystagmus is another vision disorder that is difficult to treat with currently available treatment approaches. Nystagmus is a condition in which there are involuntary eye movements causing the eyes to rapidly move from side to side, up and down, or in a circle.

The consequences can be quite significant with bilateral loss of visual acuity that affects activities of daily living, and may slightly blur vision. Currently, there are limited surgical and non-surgical treatment options, but none of these approaches has been shown to be effective. In most cases of nystagmus, the patient is given compensatory strategies to help improve function such as better lighting, large print, magnifiers, and telescopes.

To provide biofeedback therapy for accommodation (focusing) and nystagmus, an approach is disclosed in U.S. Pat. Nos. 4,533,221 and 4,660,945 to Trachtman. The methods disclosed illustrate accommodation of the eye measured by an infrared (IR) measuring technique, which is used to produce a tone to which a person may be trained to respond by lowering or driving up the pitch. The apparatus, however, requires a long-time fixation of the head during the therapy session, which renders this kind of treatment almost impossible for children, who have shorter attention spans than typical adults. Moreover, assessment of accommodation does not describe eye alignment of eyes, which is essential for treatment of persons with strabismus.

U.S. Pat. No. 5,374,193 (issued Dec. 20, 1994) to Trachtman discloses the method and apparatus for training to remain in alpha-state (when the brain emits alpha-waves) using the EEG for brain state evaluation and using EMG for muscle tension detection. Improved methods of reflected-back radiation from the eye detection incorporating a two-dimensional CCD matrix have been used.

The EMG has been used for relaxation training. Such training to remain in the alpha-state may be useful for vision improvement in adults, but much less effective in children due to their having poorly expressed alpha-waves. The application of this method requires the long-time fixation of the head to a holder. This is physically inconvenient for a person, and particularly so for a child.

Known methods of biofeedback therapy based on the restoration of visual acuity of the eye with a misaligned optical axis are only about 50% effective. This is due in part to inadequate choices of the EEG components for the brain state evaluation. The brain alpha-waves used in these techniques are poorly expressed in young children with low visual function and therefore provide no opportunity for efficient and effective analysis.

U.S. Pat. Nos. 4,533,221, 4,660,945, and 5,374,193 disclose a detection of the refraction of the eye to evaluate movements of the eye. However, these methods lack the complete description of the oculomotor function. It is impossible to detect eye alignment of the two eyes when refraction from only one eye is detected.

Thus, there remains a need in the art to address the significant problems faced by current diagnostic and therapeutic methods for ocular disorders. It would be desirable to create an objective system and method that would be usable outside of the clinical setting. Such an objective system and method would be an important tool to quantitatively measure and then subsequently treat eye alignment disorders.

### SUMMARY OF THE INVENTION

The present invention overcomes the disadvantages of previous methods and systems and offers additional benefits. Recognizing the above-mentioned challenges, “subjective” visual measures are replaced with the methodologies of the present invention. The present invention addresses the above-mentioned challenge in one aspect by providing an objective measure of strabismus control over extended periods of time. This provides the clinician with the information required to determine if treatment should be recommended or watchful waiting is appropriate.

In one aspect, the present invention can be used as a treatment approach for oculomotor disorders with a goal of decreasing the amplitude and frequency of the eye deviations. If these goals can be accomplished, it leads to improved visual acuity and function in activities of daily living. The invention would use biofeedback to present objective information about involuntary eye movements to a patient. Previous studies have shown that when supplied with this information, the amplitude and velocity of oculomotor movements can be reduced.

In another aspect of this invention a method and system are disclosed to diagnose and quantify ocular disorders, including, but not limited to, strabismus, and nystagmus. Also disclosed is a hardware diagnostic platform that collects a patient's eye tracking data during daily living and in a certain manner described herein. This eye tracking data is then used to provide new data to be used by clinicians to assess a patient, and to create an auditory biofeedback therapy that avoids the drawbacks of current technology.

Exemplary embodiments of the present disclosure relate to systems, methods, and apparatus for the treatment of ocular misalignment disorders, such as intermittent exotropia (IXT). In particular, disclosed is a system, method, and apparatus for quantification and feedback of eye deviations during normal viewing conditions. To achieve this, exemplary embodiments can employ a vision therapy system that includes a biofeedback system. One embodiment of the biofeedback system is to emit a tone when a certain threshold of deviation has been exceeded while the person is doing a visual task that they enjoy. This embodiment is hypothesized to improve patient compliance especially for younger children who have reduced attention spans compared to adults. The tone is determined by the amount of eye deviation. For example, a higher pitch tone could signify that the eye is further misaligned compared to a lower pitch tone, while no tone and/or sound would mean that the patient has proper alignment. This will help to develop voluntary convergence which is an essential first step in any vision therapy program. Biofeedback therapy will also improve fusional reserves (compensatory ability to regain normal eye alignment), as do current versions of vision therapy; however, as this is a more active method, it will help to develop those fusional reserves faster, reducing the total amount of therapy needed. This method is also non-invasively integrated with visual tasks that a person would enjoy performing to improve patient compliance.

The biofeedback and/or vision therapy system can be used either in the doctor's office or at home. In use, if a patient is given feedback so that they know that their eyes are not aligned, they will be able to voluntarily realign their eyes to resume normal binocular vision.

In one embodiment, a vision therapy system is disclosed that includes components that are worn by a patient, such as a camera and mounting system, and components that are remote from the patient, such as a microcomputer, a screen, and a power supply. The camera and mounting system may, depending on the embodiment, include a first camera, a second camera, and a headset component, such as glasses, eye spectacles, and the like.

In one embodiment, a vision therapy system, referred to as a binocular eye alignment monitor (BEAM) by the present inventors, provides a portable eye tracking platform. The software allows the user to independently track the position of each eye over an extended period in a clinical setting, and/or during daily living outside of a clinical setting. The position of each eye is then compared to normal eye alignment to detect deviations in the alignment of each eye. The data is then displayed via multiple plots. For example, a histogram of deviation of eye position deviation compared to normal eye alignment and time spent at that deviation, or the deviation over time plot, allows clinicians to understand the manifestation of the eye position deviation over extended periods of type during typical daily activities. This will allow clinicians to better treat and care for patients with eye alignment disorders.

One embodiment of the present invention for the BEAM device utilizes custom designed software and hardware as well as commercially available hardware to provide a portable and inexpensive eye tracking platform. The BEAM device and system allows the user to independently track the position of each eye over an extended period of time outside of a clinical setting. The position of each eye is then compared to detect deviations in the alignment of each eye. The data are then displayed via multiple plots including but not limited to a histogram of deviation amount, time spent at that deviation, and a deviation over time plot. This data allows clinicians to understand the manifestation of the eye position deviation to better treat and care for patients with eye alignment disorders. The custom designed hardware involves a case to hold all the parts together, including power units, screens, and cabling. It also has a headset component that is used to secure the cameras in front of the eye.

The headset component could be used to secure the cameras in front of the eyes. The headset allows each camera to be adjusted to ensure that the eye is in frame for the duration of the assessment. In one embodiment, the headset component could have swappable or adjustable parts to best evaluate individuals with varying facial anatomies.

The data are first captured by using one of several accepted methods for locating the center of the eye, commonly done by locating the circular pupil within the image of the eye, such as but not limited to a Circle Hough Transform or algorithm. The Circle Hough Transform (CHT) is a basic feature extraction technique used in digital image processing for detecting circles in imperfect images. The circle candidates are produced by “voting” in the Hough parameter space and then selecting local maxima in an accumulator matrix. It is a specialization of the known Hough transform.

Once the center of the circle is determined, this position is recorded as an X and Y (horizontal and vertical) pixel position. In the diagnostic mode, a second algorithm that is part of the process devised by the present inventors begins by converting the pixel data into an amount of rotation (ex. degrees or prism diopters, A). The first step is to smooth the whole data set using a running average filter. Then the data stream for each eye is normalized around 0 by subtracting the first value from the whole data set. This centered data is then converted to a measure of rotation using the calibration values gathered before the test began. Once the pixel to rotation conversion is completed, the process then subtracts the left eye signal from the right eye signal. The difference in eye rotation is the measured deviation amount. Once the deviation amount is calculated, two further calculations are complete. First the Inter-Deviation Interval (IDI) measures the temporal distance between any deviations if multiple occur, by subtracting the end time of a deviation from the start time of the next.

Also calculated is the deviation velocity, which is the derivative of the rotational position data. The process is similar in the therapy mode, but instead of doing the calculation in post processing, the device collects W of a second of data, and takes the average position of the points. This is then run through a similar process as the diagnostic data, without the running average filter. The amount of deviation calculated is then compared against the threshold set by the operator, and if it is exceeded continuously for more than an amount of time dictated by the clinicians, the biofeedback is emitted. In both modes the full dataset is recorded and saved.

These processes can be seen in FIGS. 5-6A and 6B for example. FIGS. 7A-7F gives the clinician interpreting the data a view of the deviation of the entire length of the test. This means that they are able to understand what was happening throughout the test time and assess any patterns or markers that may be clinically relevant. FIGS. 7A-7F give the clinician a better understanding of the amount of time that the patient's eye spent at certain deviation amounts. This will allow them to better gauge the magnitude of any ocular deviations. FIGS. 7A-7F allow the clinician to compare the data of a single patient over a series of recording sessions. This is important if the clinician is attempting to track the severity of the disorder over multiple times of day or days of the week. The heatmap aspect is to reduce the clutter that would be experienced if multiple line plots were stacked on top of one another. FIGS. 7A-7F give the clinician a finer look into the deviation over a single recording session. With the separation of the hours and minutes allowing the clinician to observe what is occurring down to the minute level in a manner that may be easier to interpret. FIG. 7F plots the Inter-Deviation Index values to allow the clinician to see any trends in the deviations both within a single session and across multiple. In one embodiment, a vision therapy system can track ocular position to provide real time feedback to the wearer to alert them to any ocular positional deviations. This can help patients with oculomotor control disorders to gain more control over their eyes and facilitate the remediate the ocular misalignment. The vision therapy system could be used for outward deviations of the eye, for inward deviations, or for deviations in both horizontal directions. The vision therapy system can be extrapolated for vertical eye deviation as well as for transient movement dysfunctions, such as nystagmus. The vision therapy system could be adapted in many ways to diagnose various types of eye movement disorders.

In one embodiment, a system and method include quantifying an ocular misalignment disorder. Included is a device recorder for quantifying ocular position over extended periods of time; calculating ocular misalignment at each time point; creating metrics for clinical use; and processing data internally, wherein the steps could be implemented either outside a clinical setting or within a clinical setting.

In yet another embodiment, a system and method for treating an ocular misalignment disorder, includes recording ocular position over extended periods of time; emitting a tone or other notable noise, music, sound, and/or vibration, to alert the user of an eye deviation to an amount greater than a predetermined threshold, predetermined by a clinician.

As previously discussed, current diagnostic techniques rely on a clinician to measure the magnitude of the deviation via a firsthand in-person examination. The present invention allows the patient to be evaluated beyond the clinical setting, as they can take the device home and use it there. As this device can be taken home, it enables long duration, continuous monitoring of a patient while performing any of a variety of visual tasks that the patient enjoys, without the need for the patient to spend large amounts of time within the clinician's office. The device is also designed to be minimally invasive, as it is a headset that holds cameras in front of the eyes. The cameras are positioned in such a way that the patient's vision is minimally obstructed. A simple user interface ensures that it is usable by anyone, and its head mounted design allows for simple data collection. This greatly reduces the impact on this patient's day-to-day vision.

The above objects and advantages are met by the presently disclosed method and apparatus. In addition, the above and yet other objects and advantages of the present invention will become apparent from the hereinafter set forth Brief Description of the Drawings, Detailed Description of the Invention, and claims appended herewith.

These features and other features are described and shown in the following drawings and detailed description. Furthermore, any combination and/or permutation of the embodiments are envisioned.

Again, other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed system and method for quantification and feedback of eye deviations and associated systems and methods, reference is made to the accompanying figures, wherein:

FIG. 1 is a diagram of an exemplary vision therapy system in accordance with one embodiment of the present disclosure;

FIG. 2 is a photo of a head camera mounting system;

FIG. 3 shows a user interface home screen and eye tracking example;

FIG. 4 shows a video transformation comparing a patient's eyes with resultant comparison;

FIG. 5 is a flowchart illustrating an exemplary process for using the vision therapy system;

FIGS. 6A-6B are flowcharts further illustrating an exemplary process of FIG. 5 for using the vision therapy system for two modalities (diagnostic and therapeutic); and

FIGS. 7A-7F show graphical outputs from the system in FIG. 1, where graphical outputs for are a graph of the difference in eye rotation (or alignment) over time are shown in FIG. 7A, a bar plot of the time spent at certain deviation amounts in FIG. 7B-7C from a typical human patient, and a heat map showing time spent at certain deviation amounts in FIG. 7D and FIG. 7E, and FIG. 7F compares the Inter-Deviation Index (IDI) between different recording sessions.

### DETAILED DESCRIPTION

Exemplary embodiments of the present disclosure include a vision therapy system that could be used for quantification of eye movements over an extended period utilizing nonintrusive methods. The vision therapy system is designed to be a portable device for use in the clinical setting and outside the clinical setting. The vision therapy system can include several hardware components as well as custom designed software to track the position of the eye over an extended period.

Current diagnostic tests for eye alignment disorders including strabismus rely on subjective or objective measurements taken over a short period of time typically only within a physician's office. In patients with constantly deviated eyes, diagnosis is easier. However, there exists a subset of eye alignment disorders that are intermittent and may not present during a clinical examination. The diagnosis and evaluation of these types of strabismus are confounded by their variability and intermittency. The current methodology measures the deviation in a clinical setting, which means it may not be a representative sample, as it requires a forced manifestation of the deviation. This creates a problem for both clinical decision-making and diagnosis as well as evaluation of treatment. An objective system that would be usable outside of the clinical setting is an important tool to help better quantitatively measure and treat eye alignment disorders.

This device also contains a novel therapeutic aspect in the form of a biofeedback system. This new therapy is designed for the treatment of ocular misalignment disorders such as intermittent exotropia. The goal of the therapy is to provide sensory feedback to the patient, so they know that their eyes are not aligned. The patient can use the sensory feedback to voluntarily realign their eyes to resume normal binocular vision. Depending on the embodiment, the device may emit a tone and/or vibration when the device observes that a certain threshold of eye deviation has been exceeded. If the user is a young child the tone may include, but is not limited to, a children's song or similar sound attractive to children. Further, the alert may be transmitted to the parent so that the parent is aware of the deviation. Such communication may be sent to a parent's electronic PDA such as an iPhone or Android, watch, or other mobile device. The threshold may be set by a clinician, user, or both.

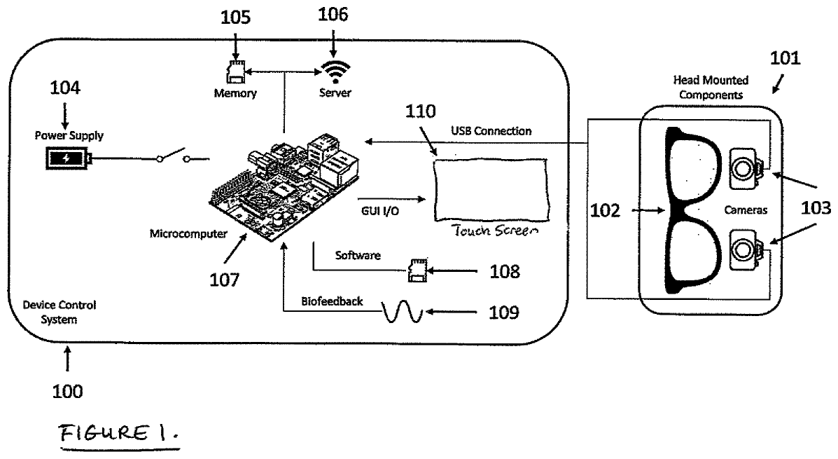

FIG. 1 shows an exemplary vision therapy system in accordance with exemplary embodiments of the present disclosure. The vision therapy system can include a device control system 100 and a head mounting system 101. The device control system and the head mounting system can be communicatively coupled to each other via wireless or wired communications such that the device control system and the head mounting system can interact with each other. In one embodiment, the device control system 100 can include a power supply 104, a memory 105, a wireless communication link 106 to the internet, a microcomputer 107 in communication with a touch screen 110. Software 108 and a biofeedback emitter 109 may be associated with the microcomputer. The biofeedback emitter 109 may include, but is not limited to a speaker, a buzzer, a visual indicator such as a blinking light, and any combination thereof. Software 108 in conjunction with microcomputer 107 facilitates the operation of the processes described herein for the practical applications and the novel methods of the present invention. Indeed the present invention provides unconventional methodologies in the treatment and diagnosis of ocular disease as shown herein and never before used in the art.

Depending on the embodiment, the device control system 100 may be attached to any portion of the head mounting system 101 as a unitary structure. In this implementation, data recorded by the head mounting system 101 may be transmitted to the device control system 100. The device control system 100 can wirelessly transmit data to a third location such as a clinician or to another remote location. Alternatively, the device control system 100 may be remote and separate from the head mounting system 101. In this embodiment, the head mounting system 101 may transmit data collected by the head mounting system to the remote device control system 100.

In one embodiment, the head mounting system 101 can include a first camera 103, a second camera 103, and a headset component 102 sized to provide a stable base on which to position the cameras and support the hardware required by the cameras. The first camera is in communication with the first microcomputer, and the second camera is in communication with the second microcomputer.

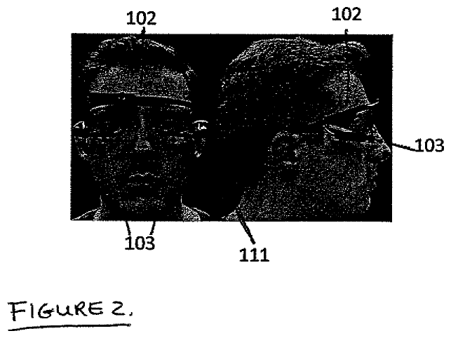

FIG. 2 shows one embodiment of the head mounting system. Depending on the embodiment a communication link 111 may connect the first and second cameras to the microcomputer. In the implementation where the device control system is not physically connected to the head mounting system, then communication link 111 is a wireless communication antenna to send data to the remote device control system 100.

Although two cameras are used in this embodiment, it will be understood that the number of cameras could vary. In one embodiment, only a single camera could be employed. Likewise, although one microcomputer is used in this embodiment, it will be understood that the number of microcomputers could vary. In one embodiment, two microcomputers could be employed.

In one embodiment, each camera is sized to capture video at 30 frames per second of the eye and the surrounding area. It will be understood that the cameras could capture video at other frames per second. The video captured by each camera is transmitted to the microcomputer. In one embodiment, each camera is an infrared light spectrum camera and is able to track the eye in everyday lighting environments.

The components of the device control system could be stored in a shell that can be placed near the wearer to minimize the burden on the wearer. An operator controls the entire device from the screen provided by the components of the device control system. The microcomputer serves to process the video from each of the cameras, and the screen is designed to display the software outputs for controlling the system. The system could include a rechargeable power supply to ensure the portability of the device.

The software can serve several distinct purposes, such as a GUI display, data collection and processing, and auditory biofeedback. The GUI allows the operator to control the system and adjust the parameters required to utilize the system. The operator could adjust various parameters, such as the position and size of a gating box, a binary mask threshold, and the size of the circle, all to ensure proper tracking of the eye. The GUI also provides visual feedback to the operator so they can properly adjust the device as needed.

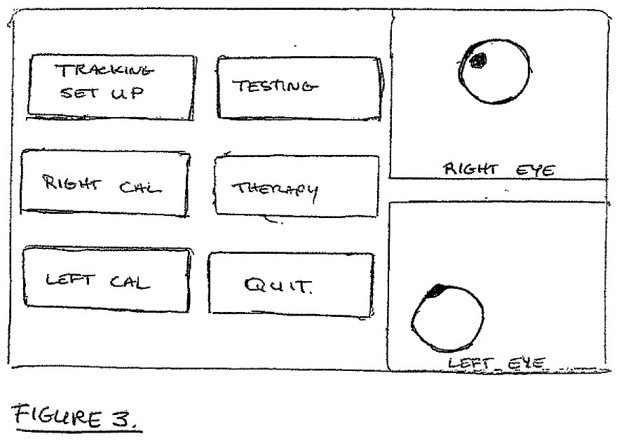

FIG. 3 illustrates two examples of the GUI screens, the left example showing the main screen, and the right showing the eye tracking being displayed. In one embodiment, the data collection and processing utilizes each frame of the video from the cameras, converts it to greyscale, and then applies a binary mask to make the image only two values. The threshold for this binary mask is set by the operator and defines the darkness cutoff for what parts of the grey image should be considered iris and pupil. The pupil absorbs IR light and will be darker than the threshold, hence in one embodiment the pupil can be set to a binary value of 1 and displayed as white. Within this embodiment, the remaining parts of the eye and surrounding tissue will reflect IR light and will be above the threshold, so will be set to a binary value of 0 and displayed as black.

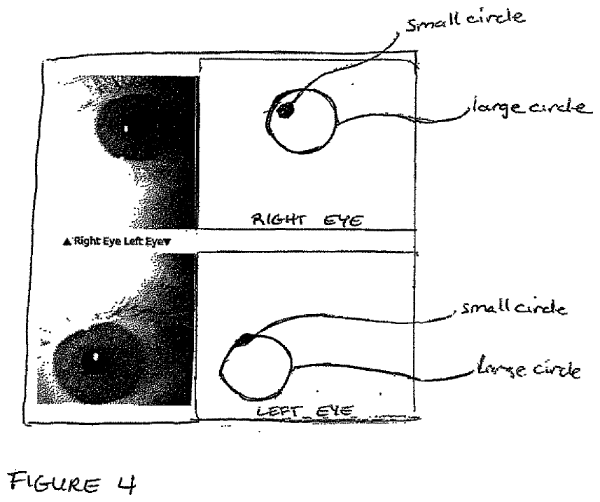

These steps can be seen in FIG. 4. A large circle shown in FIG. 4 within the gating box represents what a tracking algorithm such as, but not limited to, the CHT (Circle Hough Transform) is attempting to locate. A small circle shown in FIG. 4 within the large circle represents the center of the eye.

In one embodiment, the system employs a deviation metric calculated using the pupil centroid data. In the diagnostic mode, the process begins with converting the pixel data into an amount of rotation (ex. degrees or prism diopters, A). The first step is to smooth the whole data set using a running average filter. Then the data stream for each eye is normalized around zero by subtracting the first value from the whole data set. This centered data are then converted to a measure of rotation using the calibration values gathered before the test began. Once the pixel to rotation conversion is completed, the process then subtracts the left eye signal from the right eye signal. The difference in eye rotation is the measured deviation amount. Once the deviation amount is calculated, two further calculations are complete.

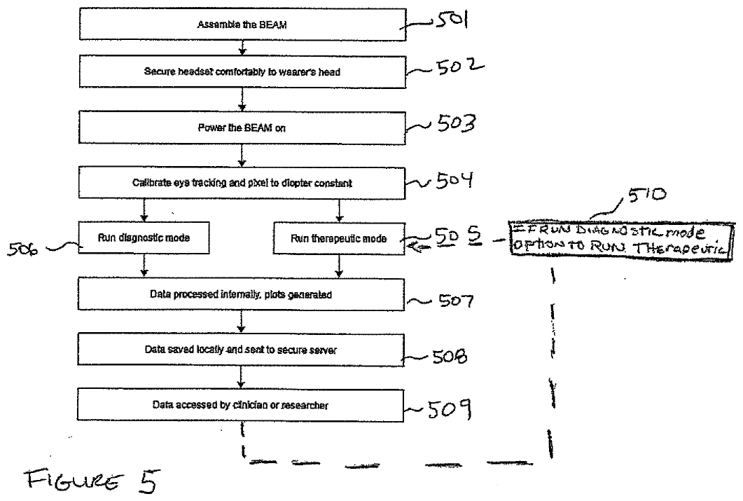

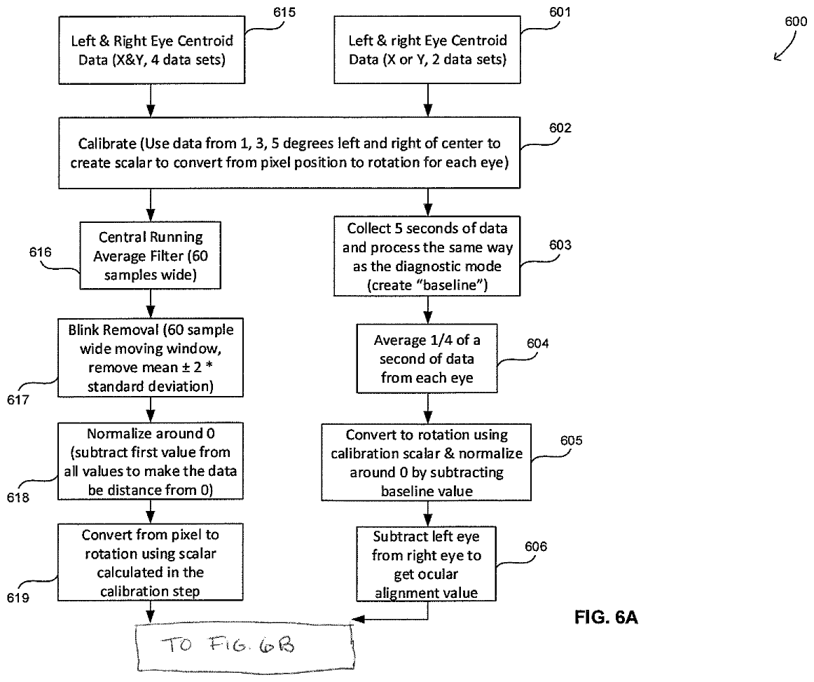

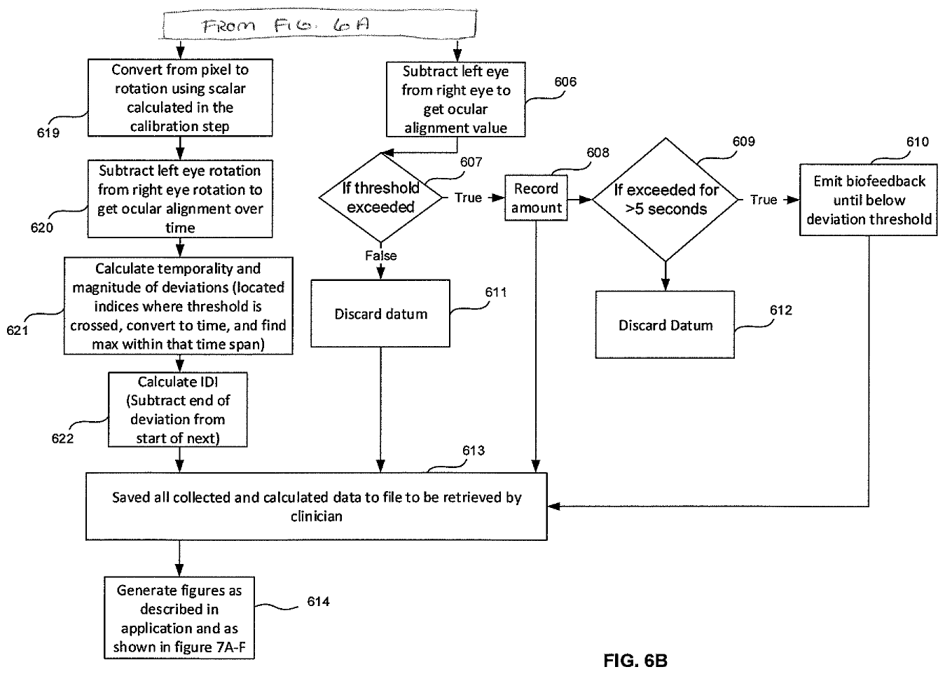

First the Inter-Deviation Interval (IDI) measures the temporal distance between any deviations if multiple occur, by subtracting the end time of a deviation from the start time of the next. Also calculated is the deviation velocity, which is the derivative of the rotational position data. The process is similar in the therapy mode, but instead of doing the calculation in post processing, the device collects a quarter of a second of data, and takes the average position of the points. This is then run through a similar process as the diagnostic data, without the running average filter. The amount of deviation calculated is then compared against the threshold set by the operator, and if it is exceeded continuously for more than an amount of time dictated by the clinicians, the biofeedback is emitted. In both modes the full dataset is recorded and saved. These processes can be seen in FIGS. 5-6A and 6B, further described herein, measures in prism diopters (PD or Δ) 1 prism diopter is equal to a prism that deflects a beam of light 1 centimeter at a distance of 1 meter from the prism. It is equivalent to 100*tan(Θ) where Θ is degrees of rotation.

The magnitude of the deviation is the number of prism diopters of misalignment existing between the eyes. The process also measures the number of deviations, in the event the individual corrects their eyes to under the threshold and then another deviation occurs. The time of each deviation and the total time deviated are the final metrics assessed.

In diagnostic mode, which depending on the implementation does not include the biofeedback, the final data set is smoothed using a running average filter. Then the process described herein in the second step calculates the magnitude, number, and length of any deviations, based on a threshold dictated by a clinician, and set by the operator in the beginning of the test.

In therapy mode, the deviation is assessed in real time. When the process detects a deviation above the specified threshold, again dictated by the clinician, a biofeedback system can emit a tone and/or vibration or other sensory cue to alert the wearer that their eyes are misaligned. The sensory cue ceases after the eye misalignment is reduced to below the threshold.

Depending on the embodiment the system may be placed in diagnostic mode and then after it has completed the diagnostic mode go into therapy mode. These steps may be performed sequentially depending on the need of the patient. This feature is shown further as an option in FIG. 5 in a block 510.

The biofeedback system is utilized in the therapy mode. In one embodiment, the auditory biofeedback system functions by emitting a tone to alert the wearer that their eyes have deviated to an amount greater than the threshold set by their clinician. Pilot data suggests that being able to hear the position of the eye improves the wearer's ability to identify and correct their deviations and make them occur less often.

When either mode is completed, and the data has been processed, the data is saved locally to the system. Several plots to visualize and understand the data are also generated. When the system is next connected to the internet, the data is uploaded to a secure, HIPAA compliant server. The data and the plots can be accessed by clinicians and researchers at any point after data collection.

Two individuals are generally preferred to properly operate the vision therapy system, although depending on the implementation the patient may perform it alone or with the assistance of a care provider or health care professional. The first individual is the wearer whose eyes will be tracked and is typically a patient. The second individual is the operator who uses the screens and the GUI to run the test. In a clinical setting, the operator could be a health professional or a therapist. Outside the clinical setting, the operator could be a caretaker of the wearer.

FIG. 5 is a flowchart of one embodiment of the process to use the visual therapy system. The first step 501 is one embodiment to assemble the vision therapy system and the second step 502 is one embodiment to secure the cameras and head set in a comfortable position for the wearer. The power is then turned on for the device as shown in step 503. After activating the software, the eye tracking is calibrated using the GUI as shown in step 504. Then the pixel position to prism diopter (PD) calibration is completed using measurements of the pixel position of the eyes at an appropriate PD, such as 5 PD, to the left and right of midline in one embodiment, with targets given by a calibration stick included with the system. Once calibrated, the operator selects either the diagnostic mode 506 or the therapeutic mode 505 and then inputs the time and deviation threshold based on instructions from the clinician.

In one embodiment, the operator selects the diagnostic mode in step 506 in an initial use of the system. The diagnostic mode can be selected in a clinical setting or outside the clinical setting. After a certain period, the data collected during the diagnostic mode is processed by the microcomputer. Alternately, the operator runs the therapeutic mode as seen in step 505. The therapeutic mode can be selected in a clinical setting or outside the clinical setting. The data is processed either internally or externally depending on the embodiment and plots generated as shown in step 507. Step 508 illustrates data saved locally either at the head mounting system, device control system, and/or at a remote server. A clinician accesses the data either in real time as the data is being collected or after the data is stored, depending on the embodiment. Step 509 illustrates one embodiment of the data being accessed by the clinician or researcher. Step 510 illustrates an optional step when the diagnostic route is taken in the system. After completion of the diagnostic steps the system may be routed to step 505 to run the therapeutic mode steps and routine as shown in FIG. 5.

FIGS. 6A and 6B illustrate a flowchart of one embodiment of the process 600 to use the visual therapy system as shown in FIG. 5. Again this flow chart is given merely as an example to illustrate the principles of the present invention and does not limit the scope of the invention to this example. Shown in FIGS. 6A and 6B is two modalities (diagnostic 615 and therapeutic 601) that function in similar but not identical manners, and both require the same set up. First the individual wearing the headset (referred to as the patient) will don the eye positional tracking glasses and be seated in a comfortable position centered on a visual display of some sort (TV, computer monitor etc.) that is 10 feet from midline away from the patient. Then a separate individual (referred to as the operator) will run the device using the provided touch screen. The diagnostic mode collects the horizontal and vertical centroid of each eye for the entire length of the test. The data are collected as pixel position. Once the data have been collected, each eye's data (both X & Y, so 4 sets in total) are smoothed using a central running average filter shown in block 616. This consists of averaging for example 30 data points to the preceding and proceeding of each value and setting the central value equal to the average of the 61 points. This reduces noise from the data. When the eye closes or blinks the signal is momentarily saturated and this can be automatically identified. The next step is to remove blinks from the data. This is done in a similar manner to the running average filter, where there is a window evaluating a fewer number of data point that systematically evaluates the dataset. The blink detection portion and removal as shown in block 617 of the process calculates the mean and standard deviation within the window.

The process identifies positional values that are greater than 2 standard deviations away from the mean in either positive or negative direction, which would be classified as blinks, equal to the mean plus or minus 2 standard deviations depending on the direction. Then each data set (X and Y for both eyes) is normalized around 0 by subtracting the first value from every value in the set as shown in block 618. This shifts the data so that it is now centered around 0. Then the data are converted from pixels to eye rotation (measured in degrees or prism diopters, which is specified by the clinician) as shown in block 619. The conversion uses the calibration data that were collected before the test began. The calibration procedure 602 has the patient look at 3 separate targets that are 1, 3, and 5 degrees to the left visual field and the right visual field from the body's midline position. The pixel position of the eye is recorded while the patient holds their eye open for 3 seconds while fixating at each degree positional target. The process then uses these pixel positions to calculate how many pixels correspond to how much rotation of each eye individually. This is done by averaging the position in each of the calibration steps, yielding 6-pixel values that correspond to known amounts of rotation. These are then averaged to 1 number, called the pixel to rotation gain value, and this is done for each eye individually.

Using this pixel to rotation conversion, the pixel position data collected during the test can be converted to a known metric, such as degrees. If the clinician decides to use the more clinical metric of prism diopters, the process will use the equation: pd=100*tan(θ) where θ is the amount of rotation in degrees. Once both eyes' data are converted into degrees or prism diopters the left eye rotation is subtracted from the right eye rotation. This subtraction gives the measure of ocular alignment as shown in block 620. Once the ocular alignment has been calculated, the first step is to assess the existence and magnitude of any deviations. The clinician determines what the threshold of deviation is for the patient. In other words, if the clinical wants the process to identify any deviations greater than 5 pd of misalignment then the threshold is set to 5.

The process evaluates the dataset and captures the indices of any time the ocular alignment crosses the threshold. These indices are then paired, as every deviation onset must have an end where it returns to a value less than the threshold assigned by the clinician. The length of deviation is then converted to time using the sampling rate of the system. If either eye position exceeds the clinician predetermined threshold, then that indicates the onset of a deviation that does not have an end to be paired with, then it will be assumed that the individual's eyes were still misaligned when the test ended and will be paired with the final value in the dataset. Within each pair (deviation) the maximum value is determined as shown in block 621. This data is then saved for retrieval by a clinician.

Also calculated at this time in block 622 is the Inter-Deviation Index (IDI). This measures the temporal distance between any deviations by subtracting the end time of a preceding deviation from the start time of the current deviation as shown in block 621. The first IDI will be the time from the start of the test to the onset of the first deviation. Once these calculations have been completed, they are all saved in block 613, and the system proceeds to figure generation in block 614.

The therapeutic mode 601 works in a similar manner, but with a few key differences. This mode runs in real time as opposed to doing the analysis post data acquisition. The first step is to generate the pixel to ocular rotation values. This is done in the same manner as in the diagnostic mode, where the position of the eyes is recorded for 3 seconds at 1, 3, and 5 degrees as shown in block 602 left visual field and right visual field measured from the body midline. Then the average pixel value is calculated for each of those recordings. This yields 6 values to generate the pixel to rotation conversion value that is required. With that metric calculated for each eye, the therapy section begins. The first 5 seconds of data are used as the central and aligned position for the test. This “baseline” as shown in block 603 is processed in the same manner as the diagnostic method described above. Every ¼ of a second, the device will take an average of the samples it has collected in that ¼ of a second as shown in block 604. The pixel values for each eye are then converted to rotation amounts using the calibration data recording during the beginning of the therapeutic session as shown in block 605. This value then has the baseline value of the corresponding eye subtracted from it as shown in block 606, which yields the amount of eye deviation from the start of the test. If the deviation amount is less than the threshold set by the clinician, then no biofeedback signal is generated as shown in block 611.

If the eye position is greater than the predetermine clinical threshold as shown in block 607, it is recorded 608, and if there are 20 consecutive measurements greater than the threshold (5 seconds) 609, the system will begin emitting the biofeedback signal 610. If the threshold is not exceeded data collected may be discarded 612. The system continues to monitor the eye alignment in this manner regardless of if the biofeedback signal is being emitted. The device may use the magnitude or length of the deviation to modulate the biofeedback signal. Once the deviation returns below the threshold then the biofeedback signal will cease. During the therapeutic mode each data sample is saved 613 so that it may be evaluated in the same manner the diagnostic mode is evaluated. Current eye trackers that are commercially available do not have the diagnostic and therapeutic aspects to them, that this device does. The process the current inventors designed is believed to add a new level of utility to these eye tracking systems and develops a new treatment modality that does not currently exist.

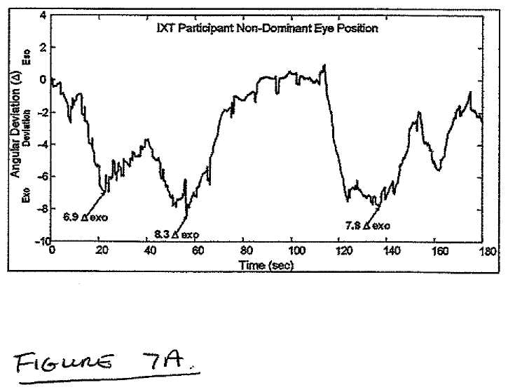

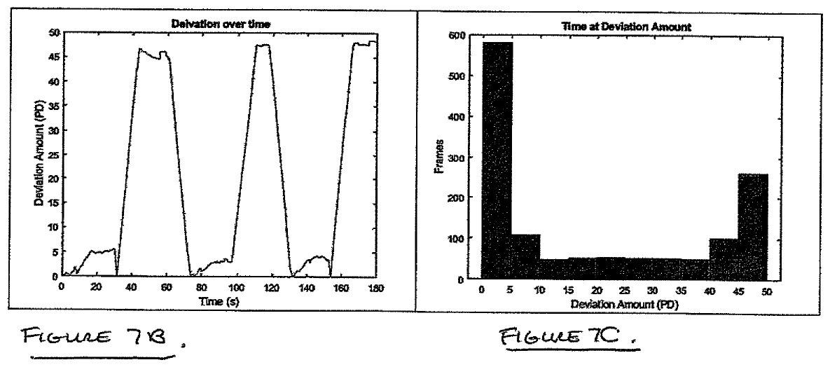

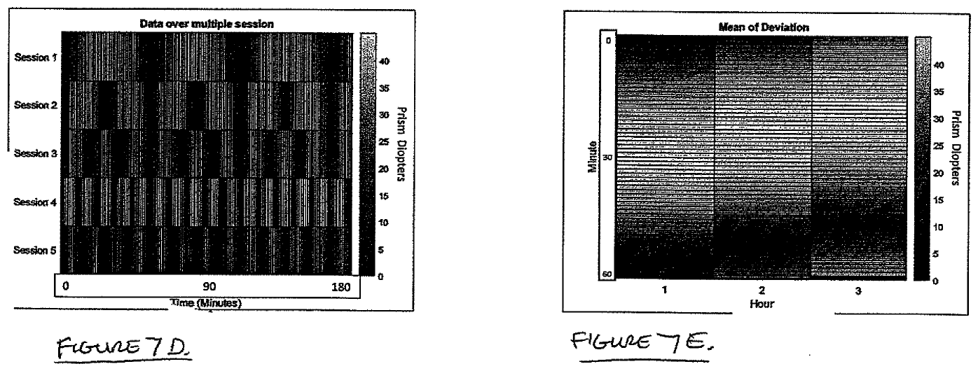

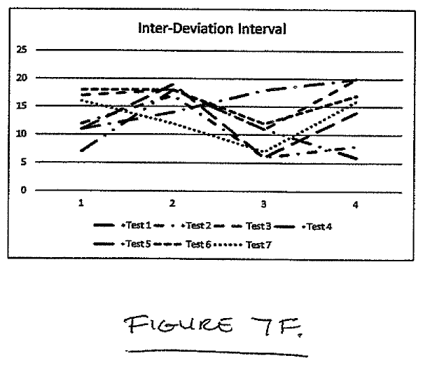

FIGS. 7A-7F illustrate embodiments of graphical outputs for the BEAM device and system. Shown in FIG. 7A-7B is a graph illustrating the difference in eye rotation (or alignment) over time. FIG. 7C illustrates a bar plot of the time spent at certain deviation amounts from a typical human patient. FIG. 7D and FIG. 7E both illustrate a heat map showing time spent at certain deviation amounts. FIG. 7F illustrates the inter-deviation interval metric.

The figures in this disclosure illustrate some embodiments and in no way limit the scope of the invention to these embodiments but are merely given to illustrate the principles of the invention. FIG. 7A illustrates how ocular misalignment over time could be displayed. Current methods do now allow for the creation of a similar plot shown in FIG. 7A as there is no method for studying ocular alignment over extended periods of time in a manner that has sufficient temporal resolution. This example also had a licensed optometrist measure the deviation prior to the test, which was measured to between 8-10 prism diopters. As noted in the figure, the device was able to measure the deviation with more specificity than the measures taken by clinicians in a normal examination.

FIG. 7B-7C contains a separate set of human data on the left (FIG. 7B), and histogram of the magnitude on the right (FIG. 7C). The histogram focuses more on the size and amount of time spent at a deviation. This allows a clinician to understand the severity of a deviation from a magnitude perspective that is quite hard with current diagnostic techniques. FIG. 7C gives the clinician a way to visualize the disorder's presentation over multiple sessions, and over time. Current diagnostic techniques only allow for the comparison of a single number, so this figure provides a great deal more information for clinical decision making.

FIG. 7D-7E gives the clinician a much finer look at the presentation of the disorders over 1 session. By giving clinicians the ability to easily understand the deviation minute by minute, this enables the clinician to assess the presentation of the disorder in a manner that the cannot currently and provides a way to assess changes over time in a more granular manner.

FIG. 7F compares the Inter-Deviation Index (IDI) between different recording sessions. This enables the clinician to assess any changes that may be occurring during sessions. Currently clinicians are limited to just the magnitude of the deviation with very limited quantitative data to assess how common a deviation is, by creating this figure, they can assess the severity of a disorder or how a clinical intervention is impacting the individual. FIGS. 7A-7B were created using actual patient data, whereas FIGS. 7C-7F were created using simulated data based on the principles of the invention.

The figures in this disclosure are some embodiments of ways to convey the data that may be clinically relevant in ways that just numbers would be unable to do. FIG. 7A illustrates how ocular misalignment over time could be displayed. Current methods do now allow for the creation of a similar plot shown in FIG. 7A as there is no method for studying ocular alignment over extended periods of time in a manner that has sufficient temporal resolution. This example also had a licensed optometrist measure the deviation prior to the test, which was measured to between 8-10 prism diopters. As noted in the figure, the device was able to measure the deviation with more specificity than the measures taken by clinicians in a normal examination. FIG. 7B-7C contains a separate set of human data on the left (FIG. 7B), and histogram of the magnitude on the right (FIG. 7C). The histogram focuses more on the size and amount of time spent at a deviation. This allows a clinician to understand the severity of a deviation from a magnitude perspective that is quite hard with current diagnostic techniques. FIG. 7C gives the clinician a way to visualize the disorder's presentation over multiple sessions, and over time. Current diagnostic techniques only allow for the comparison of a single number, so this figure provides a great deal more information for clinical decision making. FIG. 7D-7E gives the clinician a much finer look at the presentation of the disorders over 1 session. By giving clinicians the ability to easily understand the deviation minute by minute, this enables the clinician to assess the presentation of the disorder in a manner that the cannot currently and provides a way to assess changes over time in a more granular manner. FIG. 7F compares the Inter-Deviation Index (IDI) between different recording sessions. This enables the clinician to assess any changes that may be occurring during sessions. Currently clinicians are limited to just the magnitude of the deviation with very limited quantitative data to assess how common a deviation is, by creating this figure, they can assess the severity of a disorder or how a clinical intervention is impacting the individual. FIGS. 7A-7B were created using actual patient data, whereas 7C-7F were created using simulated data.

As previously discussed, the BEAM may or may not utilize a Hough Circle Transform, or other existing algorithm, to detect circles in an image. The BEAM takes each frame of the video of the eyes and applies this transform looking for circles of a certain radius that is set by the user at the beginning of the test period. When it is determined where the circle is located, the present system will record the position of the center of that circle, which will be the position of the eye in that frame. This set of positions of time is compared between the two cameras.

The process described herein then uses the position of the pupil to calculate the temporal length and magnitude of any ocular deviations from binocular fusion based on the threshold set by a clinician. The process converts the pixel data into an amount of rotation (ex. degrees or prism diopters, Δ). The next step is to smooth the whole data set using a running average filter. Then the data stream for each eye is normalized around zero. The values calculated by this process are displayed and saved for use by the clinician. In one embodiment, the software includes a custom-built graphical user interface or GUI to make the device usable by anyone.

There is also a therapeutic aspect of the BEAM device. Prior research supports that when a patient has another sensory cue of their eye position such as an auditory tone or vibration then a patient may be able to improve the motor control of their eyes. The BEAM device may utilize its eye tracking capabilities to give real time feedback to the wearer or clinician depending on the embodiment to alert them to any deviations through a sensory cue.

Again, depending on the embodiment, the BEAM device may use (2) two steps or algorithmic processes. A number of different accepted algorithms can be used to determine the center of the eye. Then another custom algorithm or process described herein uses these centers to detect the size and length of deviations. The process converts the pixel data into an amount of rotation (ex. degrees or prism diopters, A). Then smoothing the whole data set using a running average filter. Then the data stream for each eye is normalized around zero by subtracting the initial value(s) from the whole data set. This centered data are then converted to a measure of rotation using the calibration values gathered before the test began. Once the pixel to rotation conversion is completed, the process then subtracts the left eye signal from the right eye signal. The difference in eye rotation is the measured deviation amount. Once the deviation amount is calculated, two further calculations are complete. First the Inter-Deviation Interval (IDI) measures the temporal distance between any deviations if multiple occur, by subtracting the end time of a deviation from the start time of the next. Also calculated is the deviation velocity, which is the derivative of the rotational position data. The process is similar in the therapy mode, but instead of doing the calculation in post processing, the device collects % of a second of data, and takes the average position of the points. This is then run through a similar process as the diagnostic data, without the running average filter. The amount of deviation calculated is then compared against the threshold set by the operator, and if it is exceeded continuously for more than an amount of time dictated by the clinicians, the biofeedback is emitted. In both modes the full dataset is recorded and saved. These processes can be seen in FIG. 5-6.

This device provides a platform for measuring eye position over extended periods of time. The immediate applications are for the monitoring of eye position both before and after any therapeutic interventions such as rehabilitation, surgical or pharmaceutical. The first application of the current device is for exotropia, or outward deviations of the eye, but could be adjusted for inward deviations or deviations in both horizontal directions. The eye can be extrapolated for vertical eye deviation as well as transient movement dysfunctions such as nystagmus. As BEAM is designed to provide a platform for eye monitoring, it could be adapted in many ways to diagnose any type of eye alignment disorder.

The ocular alignment test runs for a predetermined amount of time set by the operation with components of the GUI included to adjust the system during the test. At the completion of the test, the data and any plots generated are saved to the device and uploaded to a HIPAA compliant, secured server for access by the clinician.

The vision therapy system allows the patient to be evaluated beyond the clinical setting, as they can take the device outside the clinical setting for example at a patient's home). The vision therapy system enables long duration, continuous monitoring of a patient, without the need for the patient to spend large amounts of time in the clinician's office.

In summary, the device of the present invention has many advantages over current technology used to address ocular alignment issues. Currently in the diagnosis of intermittent ocular misalignment disorders there are limited quantitative measurements used in the clinical setting to understand the temporal characteristics of the disorder. Clinicians will use methods like the cover test to assess the maximum magnitude of the deviation. They will assess the vergence amplitudes and levels of suppression to assess the impact on a patient's vision. Then they will also use the office control score measure to understand how well the patient can control their eyes. The procedures use analog tools that do not capture objective data and hence, the clinicians require extensive training and practice to master the ability to diagnose and treat a patient. The current conventional clinical measures are subjective as it requires the clinician to decide regarding different factors, and more importantly all these measurements require the patient to be in a clinical setting.

In contrast, the BEAM offers a significant advantage over the conventional clinical method. The BEAM is first an objective system, it is an eye tracking platform with custom written code for the detection and qualification of eye misalignment. It is designed to be used at home, where the patient can be comfortable and relaxed. The comfort of the patient's home is important to avoid “white coat syndrome” where some patients may be nervous or more or less attentive which can lead to different assessments depending on the patient.

One embodiment of the BEAM is that it can monitor the position of each eye independently for up to (4) four hours at a time. Using this data, the BEAM can then calculate how long the patient's eye was deviated above a certain threshold, the magnitude of the deviation, and provide the clinician an accurate quantitative assessment of the eye position over the test time. By giving objective measurements, the BEAM device is reducing subjectivity, whilst also providing the clinician with a more comprehensive understanding of each patient's severity beyond the clinical setting since the test can be performed for extended periods of time, allowing for better clinical decision making.

Beyond diagnostics, the BEAM device also includes a therapeutic component. The current therapeutic options are surgery or therapy. The surgery involves adjusting the position of the muscles that control lateral eye movement and is invasive. The current therapies involve strategies to improve the patient's neural control of the muscles that coordinate the eye's alignment, and often require many hours of treatment over several weeks to months to be effective.

The BEAM incorporates eye tracking into a therapeutic modality via the use of biofeedback. Prior research has suggested that if a patient can hear the position of their eyes, then they can voluntarily converge them. The BEAM emits a sensory cue (sound, vibration, visual, and the like) as previously described herein when the eyes are misaligned by more than a certain amount. The sensory cue will tell the patient their eyes are misaligned, and they must realign them to lower the pitch or make the tone stop. The tone also is determined by the amount of eye deviation so for example a higher pitch tone could signify that the eye is further misaligned compared to a lower tone, while no tone/sound/vibration or the like would mean the patient has proper alignment. This will help to develop convergence, divergence, and eye movement coordination and/or control like the current versions of therapy, but as this is a more active method, it will help to build these skills faster, reducing the total amount of therapy needed. This method and system are also non-invasive, making it a desirable first line method to treat strabismus and nystagmus. The BEAM is better for the needs of IXT as it is portable and records with sufficient temporal and spatial resolution needed for accuracy and precision to assess IXT and other binocular dysfunctions.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A method for quantifying and diagnosing an ocular misalignment disorder, comprising:
securing a head mounting system on a user, the head mounting system having at least one camera, and the head mounting system in communication with a device control system;
independently tracking and recording a plurality of positions of pupils in each eye of the user over an extended period of time in ambient lighting during the user's normal lifetime routine;
comparing positions of each eye to detect deviations in alignment of each eye;
calculating a temporal length and a magnitude of any deviations or ocular misalignment based on a threshold value to obtain a resultant value; and
displaying calculated values on a graphical user interface (GUI).

2. The method of claim 1, further includes calibrating eye position to yield value in a prism diopter.

3. The method of claim 2, wherein the displaying calculated values further includes displaying a histogram of deviation amounts and time spent at a specific deviation, and a time plot showing the specific deviation, and treating the user by altering a setting on the head mounting system for treatment of user to improve an ocular misalignment disorder.

4. The method of claim 1, further includes locating centers of pupils in each eye in an image, and comparing a set of center positions over time between each eye of the user to obtain a comparison set.

5. The method of claim 4, wherein the calculating further includes using the comparison set to calculate the temporal length and the magnitude of any deviations.

6. The method of claim 1, wherein the tracking further includes giving real time feedback to the user wearer or a clinician and alerting the user or the clinician to any deviations in eye position.

7. The method of claim 1, wherein the threshold value set is set by a clinician or the user.

8. The method of claim 1, wherein the calculating of ocular misalignment is done as a function of time.

9. The method of claim 1, wherein the device control system is remote from the head mounting system.

10. The method of claim 1, further includes implementing steps either outside a clinical setting or within a clinical setting.

11. The method of claim 1, wherein the display calculates values on a graphical user interface (GUI) is for use by an operator.

12. The method of claim 1, further comprising emitting an alert to the user, a caregiver, and/or a clinician of an eye deviation to an amount greater than the threshold value.

13. The method of claim 12, wherein the alert is selected from a group consisting of a tone, a vibration, a song, a verbal alert, a musical note, a sound, sensory cue, and any combination thereof.

14. The method of claim 12, wherein the alert is emitted at various intensities related to the magnitude of any deviation.

15. The method of claim 1, wherein the tracking further includes calibrating eye tracking and pixel to a prism diopter constant.

16. A method for therapeutic intervention of an ocular misalignment disorder, comprising:
providing a head mounting system;
calibrating eye position to yield value in a prism diopter;
tracking and recording a plurality of positions of each eye of the user over an extended period of time in ambient lighting during the user's normal lifetime routine;
comparing positions of each eye to detect deviations in alignment of each eye;
calculating temporal length and magnitude of any deviations or ocular misalignment based on a threshold value to obtain a resultant value;
displaying calculated values on a graphical user interface (GUI);
emitting a sensory cue as a form of feedback to remediate or improve ocular misalignment disorders.

17. The method of claim 16, further comprising emitting an alert to the user of an eye deviation to an amount greater than a predetermined threshold as part of a treatment of an ocular misalignment disorder.

18. A system quantifying and treating nan ocular misalignment disorder, comprising:
a head mounting system including a first camera sized to capture a first video and a second camera sized to capture a second video; and
a device control system in communication with the head mounting system, the device control system including a microcomputer sized to process the first video and the second video from the second camera, wherein the device control system outputs a data received from the microcomputer, and wherein the device control system and the head mounting system are accessible outside a clinical setting or within a clinical setting.

19. The system of claim 18, further comprising a sensory biofeedback system for emitting a tone, a vibration, a visual indication, or any combination thereof to alert the user of an eye deviation to an amount greater than a predetermined threshold.
